(19) United States
(12) Patent Application Publication
(10) Pub. No.: US 2009/0054751 A1
(43) Pub. Date: Feb. 26, 2009

(54) Touchless Sensor for Physiological Monitor Device

(76) Inventors: Bruce Babashan, Bethesda, MD (US); Vincent Luciani, Mount Airy, MD (US)

Correspondence Address: HANSEN HUANG TECHNOLOGY LAW GROUP, LLP, 1725 EYE STREET, NW, SUITE 300, WASHINGTON, DC, 20006, US

(21) Appl. No.: 11/842,992

(22) Filed: Aug. 22, 2007

(51) Int. Cl. A61B 5/02

(52) U.S. Cl. 600/324

(57) ABSTRACT

A device for monitoring heart rate and blood oxygen levels using improved pulse oximetry sensors. Pulse oximetry sensors function in either transmission mode or reflectance mode. The device of the present invention provides improved sensors functioning in transmission mode to be useful on anatomical structures with dense tissue, such as the wrist. Additionally, a combination of sensors are used to enhance the performance of monitoring devices using pulse oximetry technology. By combining sensors that function in transmission mode and reflectance mode, quality and accuracy of the monitoring device is enhanced. The data from the sensors are communicated with a microcontroller for analyzing the data. More accurate data collection translates to more accurate analysis using formulas or algorithms. The resulting analysis is conveyed to the user through a display, either digitally or in color.

### FIELD OF THE INVENTION

The present invention relates generally to devices that monitor and report physiological measurements and in particular to heart rate and blood oxygenation reporting devices.

### BACKGROUND OF THE INVENTION

Monitoring homeostasis and physiological changes that occur in a body is important to evaluating the health of a person. Pulse oximetry technology is one technology that allows for monitoring both the heart rate and blood oxygen levels. Pulse oximes sensors generally function in either transmission mode or reflectance mode. Transmission mode sensors send light across the tissue from a light emitter to a photo detector. In conventional transmission mode sensors, the light emitter and the photo detector are located across from and facing one another. The light emitter and photo detector are typically placed on either side of a thin part of the patient's anatomy, usually a fingertip or earlobe, or in the case of a neonate, across a foot, and a light containing both red and infrared wavelengths is passed from one side to the other. Changing absorbance of each of the two wavelengths is measured, allowing determination of the absorbances due to the pulsing arterial blood alone, excluding venous blood, skin, bone, muscle, fat, and (in most cases) fingernail polish. Based upon the ratio of changing absorbance of the red and infrared light caused by the difference in color between oxygen-bound (bright red) and oxygen unbound (dark red or blue, in severe cases) blood hemoglobin, a measure of oxygenation (the per cent of hemoglobin molecules bound with oxygen molecules) can be made.

In reflectance mode, the light emitter and the photo detector are typically adjacent to one another. In this sensor, the red and infrared light from the emitter travels into the tissue and is reflected back upward and is detected by the photo detector. As with transmission mode sensors, the changing absorbances of the two wavelengths due to pulsing arterial blood are measured and the measure of oxygenation can be made.

Conventional pulse oximetry devices face certain limitations. One limitation is that sensors functioning in transmission mode only function on thin vascular anatomical structures such as an earlobe or fingertip. The thinness of the tissue allows the light that is emitted to pass through the tissue to reach the photo detector. If the anatomical structure is too dense, the pulse oximeter may not function properly. This is because light from the emitter can not pass through the dense tissue and the photo detector will be unable to measure the light absorption. In addition, the conventional placement of transmission mode sensors which are typically worn on the earlobe or fingertip is not conducive to the vigorous movements of an athlete performing or engaged in activity. Another limitation is that ambient light may cause interference with both a transmission and reflectance mode sensor reading. For example, sun light which leeks to a photo detector through the edges of a poorly designed heart monitor device may cause the photo detector to erroneously register more light than that which is transmitted by or reflected by the light emitter.

Another limitation of conventional heart monitors is the method and manner in which the detected pulse oximetry information is displayed to the user. In conventional devices a digital display is employed. Such displays are acceptable when the user reading the information of display is in a static position. However, such displays of information are difficult to read when the user attempting to read the information is dynamically moving, such as during exercise. Even in the case of stationary, permanently installed monitors used with exercise bicycles, rowing machines, treadmills, etc., the conventional digital displays can be difficult to read, due to the movement of the person using the device. The embodiments of the present invention provide improved devices and methods to overcome these limitations.

### SUMMARY OF THE INVENTION

The various embodiments provide a device for monitoring physiological parameters using pulse oximetry technology. To overcome the limitation of transmission mode sensors in anatomical structures with dense tissue, embodiments herein provide an improved one-sided sensor assembly. This one-sided sensor assembly may then be used to monitor physiological parameters, such as heart rate and blood oxygen levels, through anatomical structures having dense tissues, such as the wrist.

Various embodiments herein provide a device for monitoring physiological parameters which includes sensors functioning in both transmission and reflectance mode. To improve the detection of physiological parameters, two sensor assemblies may be combined. By combining the one-sided sensor assembly functioning in transmission mode and using it simultaneously with sensors functioning in reflectance modes, a device such as a heart rate monitor may provide more accurate and robust information.

The various embodiments provide a heart rate monitor which conveys information on the heart rate of the user in the form of a relatively large color field to indicate a general range or zone for the user's heart rate. This means of conveying heart rate information is a considerable improvement over digital displays used in the past, as the user is able to determine at a glance whether or not his or her heart rate is in the desired range. The relatively small digital displays conventionally used for providing heart rate information in a heart rate monitor are quite difficult to interpret during vigorous exercise, particularly in the case of small, wrist-worn heart rate monitors when the user is moving or swinging his or her arms vigorously. Even in the case of stationary, permanently installed monitors used with exercise bicycles, rowing machines, treadmills, etc., the conventional digital displays can be difficult to read, due to the movement of the person using the device. Moreover, even in those cases where the display can be read by the user, there is little point in providing heart rate information to the resolution generally achieved by such devices, i.e. displaying the pulse rate to the nearest single beat per minute during vigorous exercise. Not only are such devices difficult to read during vigorous exercise, but the user must also calculate the desired heart rate range or zone for the exercise being accomplished, and consider whether or not the displayed heart rate number is within this zone or range.

In an embodiment, the heart rate monitor responds to these problems by providing a color display which indicates a general range or zone for the heart rate, rather than a specific number. The embodiment heart rate monitor may be configured in as a relatively small, portable device for wearing upon the wrist of the user or for carrying in the hand of the user, or may comprise a permanently installed device incorporated with a stationary exercise machine or other apparatus, as desired. The color displayed corresponds to a heart or pulse rate range, rather than to a specific number. The person using the embodiment heart rate monitor, need only exercise as required to cause hi s or her heart rate to reach the desired zone, whereupon the color field will indicate such by displaying the appropriate color. Input means may be provided with the device, enabling the user to input variables such as his or her age and gender, and/or perhaps other variables as well, depending upon the degree of complexity desired for the device.

In another embodiment, an algorithm may be programmed into the device to control the color field display in accordance with the heart-rate range or zone achieved by the user. The implemented algorithms may be any formula for calculating physiological parameter levels. The specific algorithm or formula is not particularly critical to the function of the embodiments; any one of several known algorithms, or such algorithms as may be developed in the future, may be programmed as desired into the microcontroller of the embodiment heart rate monitors. An example of such an algorithm is the Karvonen formula, which determines a target heart rate by subtracting the exercising person's age and resting heart rate from e.g. 220 (for men) or 226 (for women). The target range is between 50 and 85 percent of the target heart rate, plus the resting heart rate. An embodiment heart rate monitor may include means for the user to input his or her age in order to use the Karvonen algorithm as described above. Other variables, such as the user's sex, and perhaps other factors, may be inputted as well, depending upon the complexity of the specific embodiment of the heart rate monitor and the algorithm or formula programmed therein.

In another embodiment, communication circuits may be provided to record heart rate information over the duration of an exercise period, and download the recorded information to a computer, if so desired. The microcontroller used in the present heart rate monitor may also be programmed to provide estimates of other functions, such as calories burned during a workout, etc. The display field may include a digital time display superimposed over the color display and independent thereof, enabling the device to be used as a wristwatch, stopwatch, or timepiece if so desired. As such a digital time indication may be difficult to read during exercise, the device may indicate in some other manner, e.g. by flashing the color field display, that a predetermined exercise period or duration has been reached. Other conventional features, e.g., battery saver mode, etc., may be incorporated into the present heart rate monitor as desired. It will also be seen that the present color display field may be incorporated into other devices as well, such as depth gauges for scuba divers, altimeters for skydivers, etc., where a quickly readable display is critical.

The provision of an easily viewed color display field in an embodiment heart rate monitor also provides considerably greater versatility for its use. For example, an embodiment heart rate monitor is not limited only to use with humans who desire to have an easily interpreted view of the range of their heart rates. The embodiment heart rate monitor in its portable configuration may also readily be adaptable to use with animals. As an example, the embodiment heart rate monitor may be applied to a race horse during exercise periods. The trainer or rider can easily see the color field display provided by the present heart rate monitor and exercise the animal accordingly to achieve the desired color display, and thus the desired heart rate which corresponds to the desired level of exertion. The embodiment heart rate monitor in its portable form may be sufficiently small to be placed upon smaller animals as well (e.g., greyhounds, etc.), yet the easily viewed display permits a trainer to note the heart rate range of the animal from some distance away.

Another embodiment provides a heart rate monitor, including: a housing; a microcontroller having a heart rate algorithm programmed therein disposed within said housing; a heart rate input device communicating with said microcontroller; and a heart rate color display field disposed upon said housing, displaying one of a plurality of colors homogeneously and uniformly over the color display field according to signals received from the microcontroller and according to heart rate input processed by the microcontroller from the heart rate input device. This device further includes a user variable input device disposed upon the housing and communicating with the microcontroller. In a further embodiment, the user variable input device is configured for at least one user variable selected from the group consisting of age, gender, height, weight, and fitness activity level.

In a further embodiment, the housing includes a case configured for wearing upon the wrist of a user; the case further includes a wrist strap extending therefrom; and the user variable input device includes a rotating bezel disposed about the case. The case further includes a plurality of radially disposed electrical contacts communicating with the microcontroller; and the rotating bezel includes an internal electrical contact, selectively communicating with the plurality of electrical contacts within the case. The housing further includes a stand extending upwardly from a stationary exercise machine; and the user variable input device includes a keypad disposed upon the stand.

In a further embodiment, the microcontroller of the heart rate monitor determines which of the plurality of colors is displayed upon said color display field in accordance with a physiological parameter calculation formula such as the Karvonen formula; and the plurality of colors comprise blue corresponding to a heart rate range of from fifty to sixty percent of the base heart rate, green corresponding to a heart rate range of from sixty to seventy percent of the base heart rate, red corresponding to a heart rate range of from seventy to eighty percent of the base heart rate, yellow corresponding to a heart rate range of from eighty to ninety percent of the base heart rate, and black corresponding to a heart rate range of from ninety to one hundred percent of the base heart rate.

Another embodiment provides a heart rate monitor, including a case configured for wearing upon the wrist of a user; the case further including a wrist strap therefrom; a microcontroller having a heart rate algorithm programmed therein, disposed within the case; extending a heart rate input device, communicating with the microcontroller; and a heart rate color display field disposed upon the case, displaying one of a plurality of colors homogeneously and uniformly over the color display field according to signals received from the microcontroller and according to heart rate input processed by the microcontroller from the heart rate input device.

This heart rate monitor may further include a user variable input device disposed upon the case, and communicating with the microcontroller. Furthermore, the user variable input device includes a rotating bezel disposed about the case. Furthermore, the case includes a plurality of radially disposed electrical contacts communicating with the microcontroller; and the rotating bezel includes an internal resistor, selectively communicating with the plurality of electrical contacts within the case. Furthermore, the user variable input device is configured for at least one user variable selected from the group consisting of age, gender, height, weight, and fitness activity level.

In an embodiment microcontroller determines which of the plurality of colors is displayed upon the color display field in accordance with the physiological parameter calculation formula, such as the Karvonen formula; and said plurality of colors comprise blue corresponding to a heart rate range of from fifty to sixty percent of the base heart rate, green corresponding to a heart rate range of from sixty to seventy percent of the base heart rate, red corresponding to a heart rate range of from seventy to eighty percent of the base heart rate, yellow corresponding to a heart rate range of from eighty to ninety percent of the base heart rate, and black corresponding to a heart rate range of from ninety to one hundred percent of the base heart rate. The heart rate monitor further includes a user variable digital display disposed over the color display field.

Another embodiment provides a heart rate monitor, including a stand extending upwardly from a stationary exercise machine; a microcontroller having a heart rate algorithm programmed therein, disposed within the stand; a heart rate input device, communicating with the microcontroller; and a heart rate color display field disposed upon the stand, received from the microcontroller and according to heart rate input processed by the microcontroller from the heart rate input device. Furthermore, there is a user variable input device disposed upon the stand and communicating with the microcontroller. Additionally, the user variable input device includes a keypad disposed upon the stand. The user variable input device is configured for at least one user variable selected from the group consisting of age, gender, height, weight, and fitness activity level. Further, the microcontroller determines which of the plurality of colors is displayed upon the color display field in accordance with the Karvonen formula; and said plurality of colors comprise blue corresponding to a heart rate range of from fifty to sixty percent of the base heart rate, green corresponding to a heart rate range of from sixty to seventy percent of the base heart rate, red corresponding to a heart rate range of from seventy to eighty percent of the base heart rate, yellow corresponding to a heart rate range of from eighty to ninety percent of the base heart rate, and black corresponding to a heart rate range of from ninety to one hundred percent of the base heart rate. Further, the user variable digital display disposed over the color display field.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention.

FIG. 1 is a block diagram of the basic components and inputs thereto for the heart rate monitor.

FIG. 2 is an environmental top plan view of a first embodiment of the present heart rate monitor being worn upon the wrist of a user, showing the basic external features of the device.

FIG. 3 is a detailed top plan view of the heart rate monitor of FIG. 2, illustrating an exemplary device for inputting the age of the user to the device.

FIG. 4 is a top plan view of the heart rate monitor of FIG. 3 with the display removed, illustrating an exemplary internal mechanism for inputting a variable to the microcontroller of the device.

FIG. 5 is a perspective view of a stationary treadmills exercise device incorporating an alternative embodiment.

FIGS. 6A is a perspective view which illustrates an embodiment.

FIG. 6B is a detailed perspective view of display units relating to an embodiment.

FIG. 6C -6D are detailed perspective view of a compartment relating to an embodiment.

FIG. 6E is a perspective and system view which illustrates an embodiment.

FIG. 6F is a perspective view which illustrates an embodiment.

FIG. 6G is a detailed perspective view of a compartment relating to an embodiment.

FIG. 6H is a perspective view illustrating an embodiment.

FIG. 6I is a perspective view of a sensor assembly relating to an embodiment.

FIG. 7 is a graph showing hemoglobin oxygenation versus wavelength of light related to an embodiment.

FIG. 8A is a cross-sectional view of a sensor assembly illustrating an embodiment.

FIG. 8B is a cross-sectional view of a sensor assembly illustrating an embodiment.

FIG. 8C is a table showing example combinations of sensor wavelengths.

FIG. 8D is a cross-sectional view of a sensor assembly embodiment.

FIG. 8E is a diagram of an embodiment in position on a subject.

FIG. 8F is a cross-sectional view of a sensor assembly embodiment.

FIG. 8G is a cross-sectional view of a sensor assembly embodiment.

FIG. 8H is a cross-sectional view of a sensor assembly embodiment.

FIG. 9 is a circuit diagram illustrating an exemplary circuit of an embodiment.

FIG. 10 is a process flow diagram embodiment of an exemplary method.

FIG. 11 is a process flow diagram embodiment of an exemplary method.

FIG. 12 is a detailed view of certain components of an embodiment.

FIG. 13 is a table of exemplary ranges and composite colors for various pulse rates and the relative pulse width for each of the primary color illumination sources.

### DETAILED DESCRIPTION OF THE PREFERRED EMBODIMENTS

The various embodiments will be described in detail with reference to the accompanying drawings. Wherever possible, the same reference numbers will be used throughout the drawings to refer to the same or like parts.

As used herein, the terms “about” or “approximately” for any numerical values or ranges indicates a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

The various embodiments may include a device for monitoring physiological parameters, such as heart rate, having a large color display field for displaying ascertained information or measurements, such as heart beat frequency range, of a user. The various embodiments may further include a monitoring physiological parameters, such as heart rate and blood oxygen levels, using pulse oximetry technology. This device may be constructed as a relatively small and portable device worn on the wrist or other area of the body or face (e.g., sunglasses) of the user, or as a larger device temporarily or permanently installed in a stationary exercise machine (e.g., treadmill, rowing machine, etc.).

It has been recognized for some time that the degree of elevation of heart rate during exercise is an indication of the level of exercise being performed. More recently, studies have determined that the greatest benefit from exercise is achieved when the exercise is performed to elevate the heart rate to a specific predetermined range, and held in that range for the duration of the exercise. More specifically, it is desired that the heart rate be raised gradually into the desired range by a series of warm-up exercises, and allowed to drop back gradually to its normal rate by a series of cool down exercises. The greatest benefit to the person involved, and the least stress and strain on the heart, is achieved when exercises are performed according to this philosophy.

With the increasing popularity of various fitness training and exercise programs, more and more armature and professional athletes are paying greater attention to specific heart rates recommended by their trainers and other programs. Technology has resulted in the development of the heart rate monitor, comprising an electronic device which detects the pulse of the user and provides a readout of the user's pulse rate. Various principles have been developed for detecting the pulse of a person using such a device, e.g., the tonometer and oximetry principles, as well as invasive means which are impracticable in a heart rate monitor for exercising persons.

The great interest in this subject by those in the medical field has resulted in the development of a number of different formulas for determining optimum heart rate for any given condition or level of exertion. For example, the Karvonen formula for determining optimum heart rate is one such formula which has been known and used for some time by those who are knowledgeable in the field. The Karvonen formula determines a target heart rate by subtracting the exercising person's age and resting heart rate from an initial number, e.g., 220 (for men) or 226 (for women); other numbers may be used. The target range is typically in a range between 50 and 85 percent of the target heart rate, plus the resting heart rate. The target range may vary from this exemplary range, depending upon the specific exercise program being used. The Karvonen formula is well known, and is used by perhaps the great majority of exercise programs which specify target heart rates during exercise. Other formulas for approximating optimum heart rate during exercise have been developed, as well as stress tests for determining heart rate.

Conventional heart rate monitors with digital pulse rate displays may provide indications of the optimum or target heart rate using the Karvonen or other formula. However, the display means of these conventional monitors always use digital means. Such digital displays of heart rate, and/or target rates, do not provide for ease of reading the display under most conditions of use. For example, these small digital displays are difficult to read when a user is jogging, moving his arm relatively rapidly and producing jarring motion as a result of rapid impact of his feet with the running surface. This is all the more true in various other forms of exercise, e.g. rowing, calisthenics, etc., where arm motion does not position a wrist mounted devices for reading a display thereon. Even when using stationary treadmill type devices, it can be difficult to read a relatively small digital display provided thereon. Moreover, it is not critical that an exercising person establish a precise heart rate, but rather that the exercise maintain a heart rate within a desired range, e.g. in accordance with the Karvonene formula and other formulas which approximate a desired heart rate during exercise.

In solving these problems, the embodiments provide a heart rate monitor which may displays the general range of the user's heart rate by means of a color display. In exemplary embodiments the heart rate monitor may comprise of a display (either portable or permanently installed on an exercise device or the like, as desired) and input means for setting basic variables (e.g., user's age and gender) into the device. Other exemplary embodiments may include means for inputting additional variables in various ways. An embodiment heart rate monitor preferably may provide an easily viewed field which displays a uniform color homogeneously across a substantial portion of the field, enabling a user to determine, just by a glance, which heart rate range or zone he is in at the moment. Different colors may signify different ranges, e.g., blue for cool down (or warm-up), red to indicate “fat burning,” black to indicate the “dead zone” for trained athletes who need to reach a higher level of cardiovascular activity, etc. In alternative exemplary embodiments, additional input means may be provided to allow the user to adjust the color display depending upon the fitness level of the user and the type of activity to be performed.

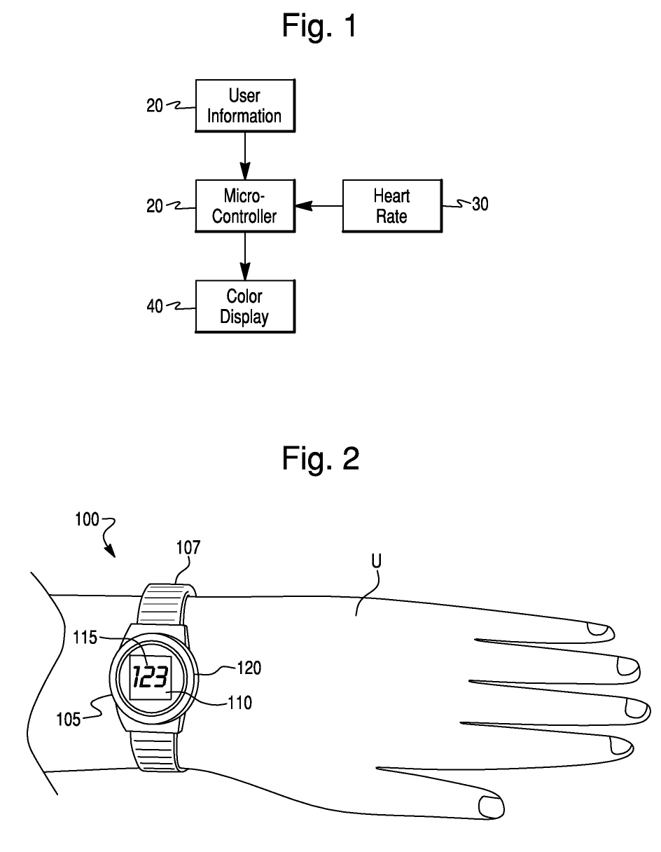

FIG. 1 illustrates the basic components of the various embodiments and their relationship to one another. The central component of the various embodiments may be a microcontroller 20, which receives input from two sources, i.e., a conventional transducer or input device 30 which measures physiological parameters, such as heart rate of the user, and a user input device 10. The microcontroller 20 may then process this information and control an easily viewed color display field 40, with the color displayed being in accordance with the heart rate measured by the heart rate transducer 30.

The microcontroller 20 may be conventional, with various such devices being available in the marketplace for carrying out the required functions of the various embodiments, i.e., measuring a pulse frequency and controlling a color display in accordance with the frequency detected. The inventive concept may comprise the use of an easily viewed color display to indicate a general range of heartbeat or pulse frequency. The microcontroller may be configured to interface with various computer devices, e.g., a personal digital assistant (PDA) device, etc., in order to record information for later review. The microcontroller 20 may be programmed with any one of a number of known formulas or algorithms for determining the optimum heart rate of a person during exercise. In the example cited herein, the Karvonen formula is used.

The Karvonen formula comprises the calculation of a target heart rate, from which a heart rate reserve range is calculated. A constant is initially provided, with the constant being different for men and women. For men, this constant is generally set at 220, and for women, 226. The embodiment heart rate monitor may provide for user input for the sex or gender of the user, in order to provide the proper constant. Once the constant has been determined, the user subtracts his or her age and his or her resting heart rate from the constant, to provide a base heart rate number from which maximum and minimum heart rates during exercise are calculated. The respective maximum and minimum heart rates are generally eighty five percent and fifty percent of the base number, plus the resting heart rate.

As an example of the above, a thirty year old male with a resting heart rate of seventy, may subtract his age and resting heart rate from the initial constant, i.e., 220−30 −70=120. The person may then multiply this result (120) by fifty percent and eighty five percent and add his resting heart rate to each result, to arrive at his respective lower and upper desired heart rates during exercise. Thus, the lower heart rate limit would be (120×0.5)+70=130, and the upper heart rate limit would be { 120 x 0. 8 5) +7 0 =1 7 2. The microcontroller 20 of the present heart rate monitor may automatically calculate the above numbers, once the user has entered his age and gender into the device. The resting heart rate of the user may be determined automatically by the heart rate transducer 30.

The heart rate transducer or input device 30 may include any of a number of known devices and/or principles of operation. A basic means of electronically detecting heart or pulse rate was developed by Willem Einthoven in 1906, with many pulse rate detectors using the same principle of operation today. Other principles and devices, e.g. plethysmography using an optoelectronic transducer, Doppler ultrasonography using a piezoelectric transducer, etc., may be used as desired for the heart rate transducer 30.

Once the microcontroller 20 has received the appropriate heart rate signals from the heart rate input transducer 30, the microcontroller 20 may then provide an appropriate signal to the color display field 40. The color display 40 may display a color in accordance with the heart rate frequency detected by the heart rate transducer 30, as processed by the microcontroller 20 according to the algorithm or formula programmed therein. The optimum display may be a color display disposed uniformly and homogeneously over a substantial portion of the color display field 40 to provide an easily viewed and interpreted indication of the corresponding general heart rate range of the user. The use of an easily viewed color field 40 allows a user of the embodiment heart monitor to determine his or her general heart rate range at a glance without needing to stop the exercise for a short period of time in order to read and interpret a relatively small digital display, as is conventionally provided with heart rate monitors.

Examples of the colors and corresponding heart rate ranges with which the present heart rate monitor might be programmed are provided below. In accordance with the exemplary Karvonen formula described further above, the user of the present device desires to maintain his or her heart rate within some predetermined range, e.g., between fifty and eighty five percent of the base heart rate number. The user may begin an exercise session with a warm-up period, during which the body is warmed up relatively slowly, muscle groups are stretched, and the heart rate slowly increases. This relatively “cool” exercise zone, comprising a heart rate between fifty and sixty percent of the base heart rate number, may be programmed to provide a blue color or tint distributed homogeneously and uniformly over a substantial portion of the color display field 40. Thus, the exercising person using the present heart rate monitor may need to only glance at the display 40 to determine whether he or she is working at the desired level. Once the relatively cool “warm-up” period has been completed, the exercising person may exert himself or herself somewhat more strenuously, thus elevating the heart rate to a somewhat higher level. The desired heart rate during this period may be between sixty and seventy percent of the base heart rate number, and may result in a green heart rate display field 40 to indicate a desired level of performance or exertion.

In many instances, the exercising person may wish to reach a higher, anaerobic exercise state or level, in which the muscle groups are exercised more strenuously and the heart rate is increased correspondingly. This heart rate level may be between seventy and eighty percent of the previously calculated base heart rate, and may result in a red color being displayed on the color display area 40, to indicate a “fat burning” exercise level. Even higher levels of exercise may result in other colors, e.g., a yellow or “caution” range for a heart rate between eighty and ninety percent of the base heart rate, and black when the heart rate exceeds ninety percent of the base rate. These colors are exemplary, and other colors may be programmed into the device as, desired. For example, a trained marathon runner may exert himself or herself to a reasonable level with a relatively low heart rate, and not develop his or her abilities further. This level of exercise is called the “dead zone” by many trainers and advanced athletes, as it does not provide the level of physical training they desire. The present embodiment heart rate monitor may be programmed to provide a black display when this level is reached, if so desired.

The display field 40, with its easily viewed and interpreted color display, may enable an exercising person to note whether he or she is in the proper activity range, even though considerable body movement is likely occurring which would preclude the ability to read a small digital display. Persons who normally wear corrective lenses, but remove them for exercise, will find the present monitor to be particularly useful. Also, the ability to program the device to provide different colors in the display for different heart rate activity levels, also provides for those persons who may have some degree of color blindness. A common form of color blindness is difficulty in distinguishing red and green. Accordingly, different colors may be used, e.g., blues, yellows, and/or perhaps oranges or other colors somewhat removed from the center of the red area of the spectrum, etc., as desired. In addition, further information may be provided by pulsing or flashing the display to attract the user's attention and/or to indicate some other condition or information.

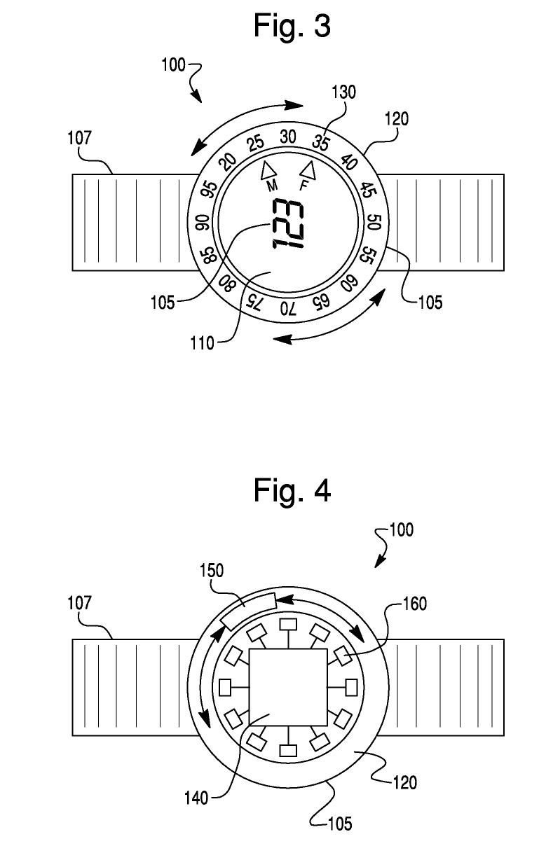

FIGS. 2 and 3 of the drawings provide top plan views of one embodiment of the present heart rate monitor invention, comprising a wrist mounted or attached heart rate monitor device 100, similar in configuration to a conventional wristwatch. The wrist mounted monitor 100 may include a housing or case 105, with a wrist strap 107 extending from each side thereof for conventional attachment of the device 100 to the wrist of a user U. The case 105 may contain the various components shown in the flow chart of FIG. 1, i.e., the microcontroller 20 and heart rate transducer 30. Alternatively, the transducer 30 may be located along the wrist band 107 or elsewhere on the body, with suitable communication between the transducer 30 and microcontroller 20 being provided. For example, this communication may be via wire connections or wirelessly.

The easily viewed color display field 1 10 may be disposed upon the outer surface of the case or housing 105, where it may be clearly visible to the user U wearing the wrist mounted monitor 100. The color display field 110 preferably may encompass the majority of the face of the case or housing 105, in order to provide the desired color surface area for ease of viewing by the user U. Various means of providing the uniform color display desired in the present heart rate monitor invention, may be used. Several color illumination sources may include: light emitting diodes (LED); electro-luminescence display, liquid crystal display (LCD) and others. For example, where relatively high electrical power consumption is not a concern, a matrix or array of pixels as used in flat screen television screens, or LEDs, may be used as desired. The technology also exists to provide color in a liquid crystal display, particularly by incorporating a stacked array to provide spectral diffraction to produce the desired color effects. Reflective LCD displays may also be used, and require less electrical power than do the other technologies noted above. Alternatively, an electromechanical display may be constructed, utilizing a small display band having the desired display colors applied to various areas thereof. The band may be rolled from end to end, with the exposed central area passing beneath the window of the display field 110. Movement of the band may be accomplished by micro-size electrical motors, or more economically by small solenoids which actuate an escapement mechanism at each roller. In an embodiment, this system requires no electrical power whatsoever when the band is stationary.

The forming of the color display field 110 from a large number of relatively small elements, generally as described above, may enable the programming to change the color, shading, or brightness displayed upon some of the elements to contrast with the remainder of the color field. Thus, a supplementary message may be superimposed upon the primary uniform color display field, if so desired. Such a supplementary message may be in the form of a digital display 115, as indicated in FIGS. 2 and 3, or some other display format, as desired. It is not intended that such a digital display provide crucial information relating to heart rate during an exercise period. This function is accomplished by the easily viewed color display field 110. In fact, the digital display 115 is not required with the present embodiment heart rate monitor, but may be provided optionally if so desired. The digital display 115 may provide the time, or perhaps a time interval for the exercise session or portion thereof, or an estimate of calories burned, etc., as desired. Conventional controls, e.g. a rotating stem or button (not shown) as used to set and adjust the time in conventional wristwatches, may be provided to adjust, activate, and/or deactivate the digital display 115 as desired.

Formulas or algorithms used for determining the optimum heart rate of an exercising person may require the input of certain variables which are dependent upon characteristics of the exercising person. Such variables may comprise the person's age, sex, height and weight, and fitness level, and/or other parameters. For example, the Karvonen formula takes into account a person's age and gender, as well as his or her resting heart rate. The resting heart rate may be determined automatically by the present heart rate monitor, as noted further above. However, the other parameters must be entered into the device by the user. Accordingly, a user input device 120 may be provided in the wrist mounted heart rate monitor 100 of FIGS. 2 and 3. The user input device 120 may include a rotating bezel which surrounds the display area 110, and generally defines the circumference of the case or housing 105. The bezel 120 may preferably include a series of numbers 130 thereon which correspond to the age of the user, and separate index marks for males and females to accommodate their different initial constants.

A person using the present heart rate monitor 100 of FIGS. 2 and 3, may only need to rotate the user input bezel ring 120 to align the appropriate age number 130 thereon, with the corresponding index mark “M” (males) or “F” (females), as appropriate. The device may automatically detect the person's resting heart rate when the device is worn while the user is at rest. This is all the information needed for the device 100 to calculate the various heart rate ranges desired during exercise for the person using the present device 100, in accordance with the Karvonen formula. Alternative formulas or algorithms which take into account other factors may be programmed into the present device in lieu of the Karvonen formula if so desired, with the user input controls being marked and indexed accordingly. It will be seen that other means of entering user variables, e.g., a series of pushbuttons, rotary knobs, etc., may be incorporated with embodiments of the present device, if so desired. Such setting and adjustment buttons and knobs are conventional, and are well known in the field of controls for miniaturized equipment.

FIG. 4 is an illustration of the internal configuration of an embodiment wrist mounted heart rate monitor 100, showing an exemplary electrical contact system for programming the microcontroller 140 contained therein. The internal volume of the case 105 may contain a plurality of electrical contacts 160 therein, disposed in a radial array immediately inside the circumference of the case 105. These electrical contacts 160 may communicate electrically with the microcontroller 140 disposed within the case 105. An electrical resistor 150 may be disposed within the ring comprising the rotating user input bezel 120. As the user rotates the bezel 120, the resistor 150 comes into electrical contact with different ones or pairs of the electrical contacts 160 within the case or housing 105, thereby providing a signal(s) to the microcontroller 140 as to the appropriate age and sex or gender of the exercising person to be used for calculating the base heart rate of the user and the corresponding calculations of the desired heart rate ranges for that user during exercise. The color output of the display area 110 may be adjusted accordingly during exercise, as described further above.

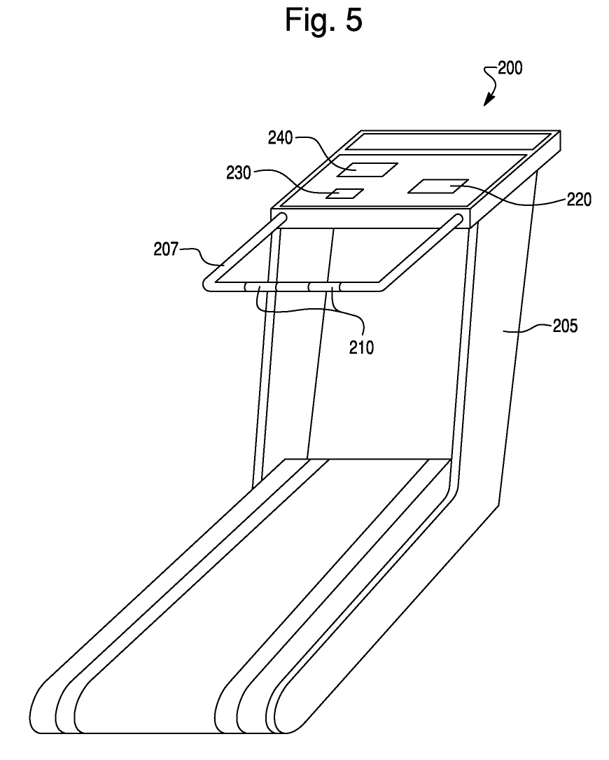

FIG. 5 provides a perspective view of an alternative embodiment of the present heart rate monitor device, wherein the device is permanently installed within a stationary exercise machine. The exercise machine illustrated in FIG. 5 shows a treadmill 200, but other types of exercise equipment, such as, rowing machines, exercise bicycles, weight machines, etc., may be used as desired. The treadmill exercise machine 200 of FIG. 5 may include a stand 205 having various input controls and displays thereon. A handlebar 207 extends from the stand 205, with the handlebar 207 providing support for the user as well as a pair of handgrips 210 which may include conventional heart rate transducer devices therewith. Other body contact means incorporating heart rate transducer devices may be incorporated as desired. The heart rate of the person using the exercise machine 200 may be received by the handgrips 210, and transmitted to the microcontroller 20 (not shown, but essentially the same as that used in the embodiment of FIGS. 1 through 4) for processing of the signal.

The stand 205 may include a conventional display 240 indicating distance covered and which may display additional information, e.g., estimated calories burned, etc. A conventional keypad 230 may be provided for the user to input information (user variables, etc.) as desired. The keypad 230 may be used to enter the exercising person's age, gender, and resting heart rate, as well as other information, e.g., height and weight, etc., as required by the particular program or formula being used with the machine 200. An easily viewed color display field 220 may be also provided, with the display 220 being driven by the microcontroller 20 (Illustrated in FIGS. 1-4)not shown) according to the programming of the microcontroller 20 (illustrated in FIGS. 1-4), the data entered using the keypad 230, and the heart rate of the user as detected by the handgrip transducers 210. The display 220 of the exercise machine 200 may utilize the same technology as described further above for the wrist attached heart rate monitor device 100, depicted generally in FIGS. 2 through 4. As the exercise machine 200 may be stationary and receives electrical power from a remote source (e.g., 115 or 230 volt ac electrical power), the power consumption of some of the technologies noted, e.g., LEDs and backlit displays, is not a concern.

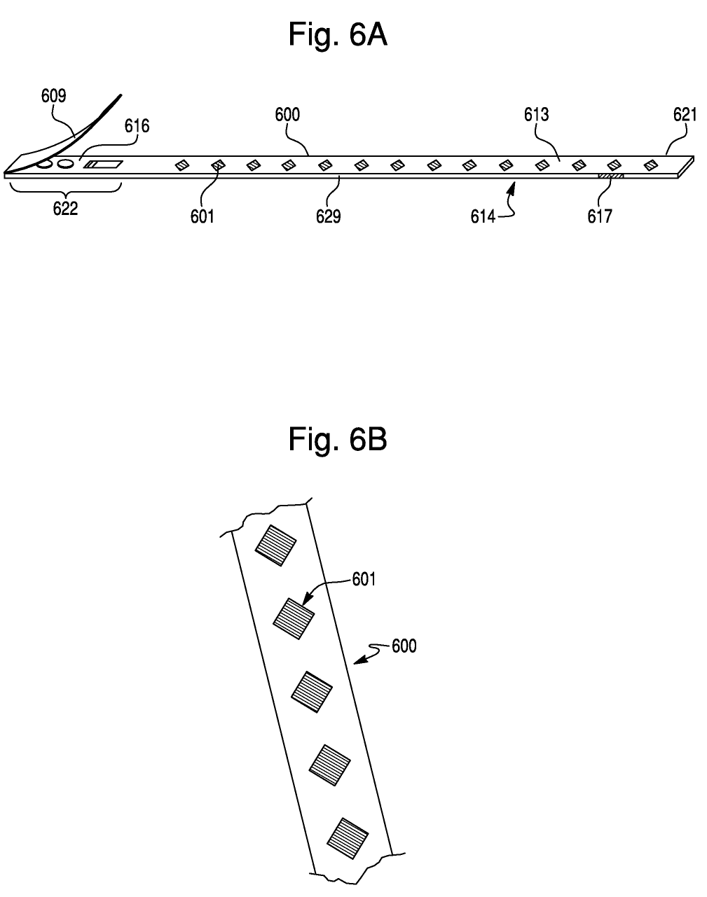

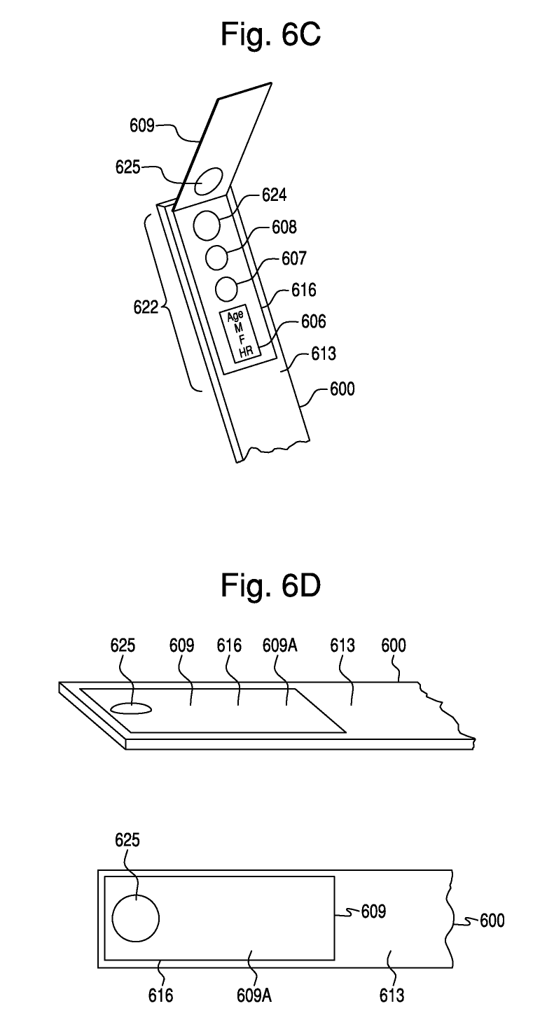

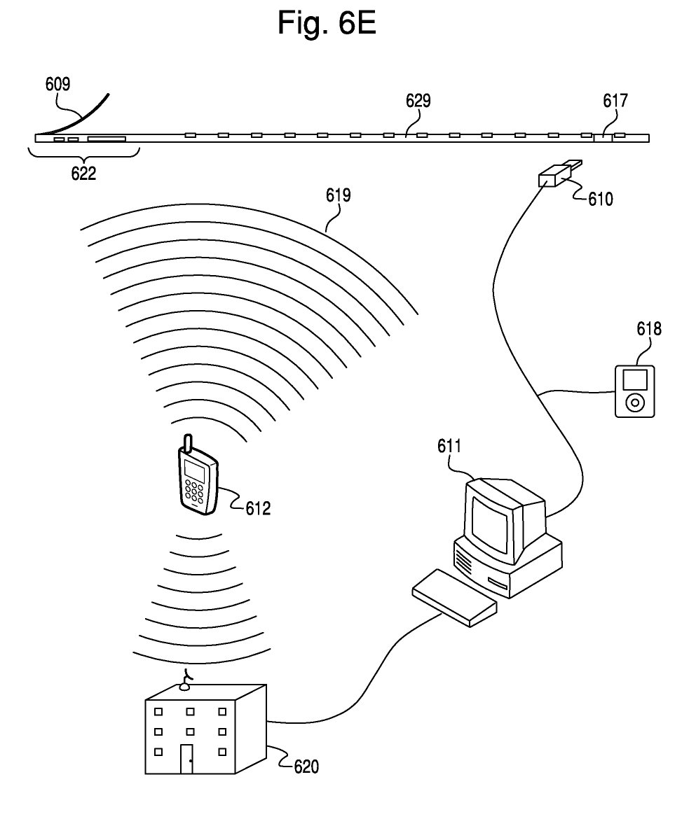

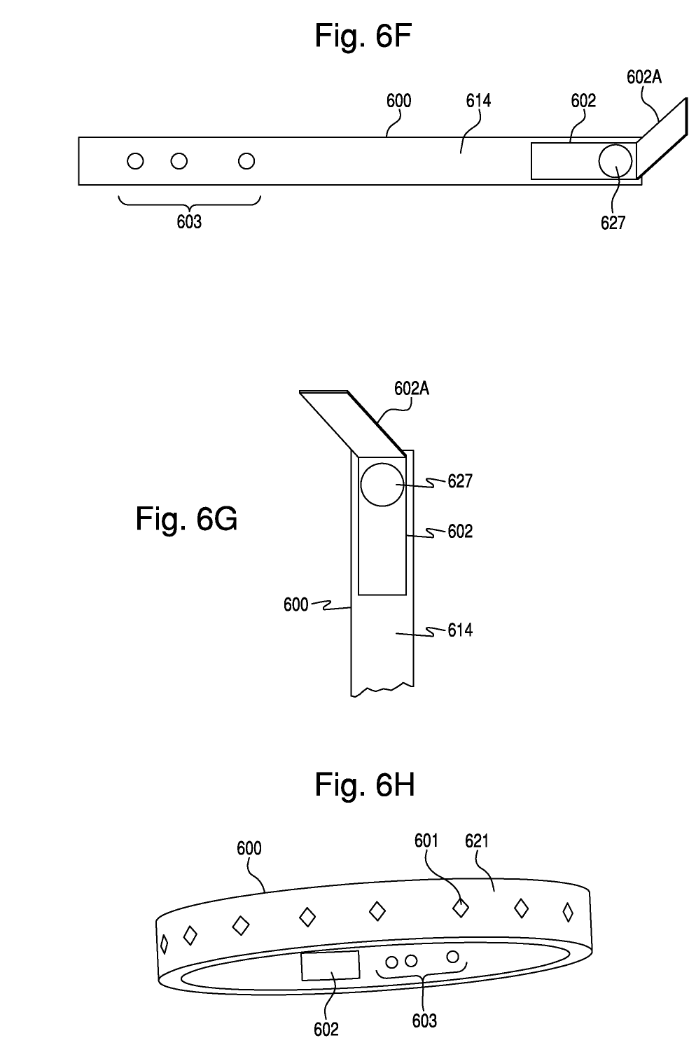

FIGS. 6A illustrates the top view of an exemplary embodiment of a portable heart rate monitor 600 which can be worn on the wrist as a bracelet. This heart rate monitor 600 may include a casing 621 with a top surface 613, a bottom surface 614 and a side surface 629. The top surface 613 of the heart rate monitor 600, as shown in FIG. 6A, may include display units 601 and a compartment 616 which houses a user input device 622. The side surface 629 may include an input/output port 617.

On its top surface 613, the heart rate monitor 600 may include display units 601. These display units 601 may be capable of receiving information from microcontroller 20 (illustrated in FIG. 1) and conveying that information to the user in different forms, such as in color or in digital form. FIG. 6B illustrates a detail view of the display units 601. This illustration shows a segment of the heart rate monitor 600 which includes an array of display units 601 set along the heart rate monitor's 600 long axis. Based on the information received from the microcontroller 20 (illustrated in FIG. 1), these display units 601 may illuminate in different colors to convey information to the user.

The design of the display unit 601, as illustrated in FIGS. 6A and 6B is only an example and other designs may be used. In the embodiment, as illustrated in FIG. 6A and 6B, the heart rate monitor 600 may contain several individually located display units 601 along the long axis of its top surface 613. This configuration may allow the user of the heart rate monitor 600 to easily read the findings and measurements from any viewing angle. Alternatively, there may be one display unit 601 on the top surface of the heart rate monitor 600 covering a portion or a majority of its top surface 613 while conveying information to the user.

In other design alternatives the display unit 601 may be connected to gem stones in a jewelry piece, placed in patterns or have colors that would compliment the user's apparels. In addition, the display unit 601 may use any color illumination source technology currently known in the art of displaying information to the user. For example, the display unit 601 may contain a light emitting diode (LED) to convey information received from the microcontroller 20 (illustrated in FIG. 1) to the user. Alternatively, the display unit 601 may contain a liquid crystal display (LCD), electronic fluorescent (EFD) or electro-luminescent display (ELD) or other display technology known in the art to convey information received from the microcontroller 20 (illustrated in FIG. 1) to the user. The information conveyed using these technologies may be by light or digital. Information conveyed to a user through, for example, LED lights may be in the form of color lights, constant lights, blinking lights, or chasing lights. Alternatively, the display unit 601 may communicate findings to a user using modes other than light or digital display. It may communicate the findings to the user using sound, touch (vibration) or other known communication methods.

In an embodiment, as illustrated in FIG. 6A and 6C, on the top surface 613 of the heart rate monitor 600 there may be a user input device 622, housed in a compartment 616. The compartment 616 may included a sleeve cover 609 to protect its interfaces. This user input device 622 may include an interface to input the personal information of the user, such as age, gender, height, weight and fitness activity level. As illustrated in the detailed FIG. 6C, the user input device 622 may include a manual user interface which may further include input controls 607 and 608. Using these manual input controls 607 and 608, the user may enter data into the heart rate monitor 600. The user input device 622 communicates the inputted information to the microcontroller 20 (illustrated in FIG. 1) which in turn may display it on the display 606. The user may then verify or correct the entered data based on the information displayed on the display 606. Additionally, the user input device 622 interface may include a variety of user interfaces to receive personal information. These interfaces may include a microphone 624 to allow a user to input data into the heart rate monitor 600 using sound, such as his voice and speech recognition software.

FIG. 6D illustrates a detailed view of compartment 616 and sleeve cover 609. The compartment 616 may be accessible through a sleeve cover 609 which may cover and protect the user input device 622. FIGS. 6D illustrate the compartment 616 when it is covered by a sleeve cover 609. This sleeve cover 609 may be connected to the heart rate monitor 600 at one end and may be pulled up from its opposite end to expose the user input device 622. Once data is entered, the sleeve cover 609 may be laid down and anchored to the body of the heart rate monitor 600 to effectively cover and protect the user input device 622. The sleeve cover 609 may be made from the same material as the casing of the heart rate monitor 600 or may have other material to allow for design and construction flexibility.

In heart rate monitors 600 where a microphone 624 is used to input information into the device, the sleeve cover 609 may include a microphone cover 625 on the top surface 609A. This is shown in FIGS. 6D. This microphone cover 625 may allow sound to reach the microphone when the sleeve cover 609 is in a closed position, hence allowing the user to program the heart rate monitor 600 without opening the compartment 616. In alternative embodiments, the microphone cover 625 may also protect the microphone 624 from external harmful elements such as water and dust, while allowing sound to reach the microphone 624 for voice command.

A user may input personal data into the heart rate monitor 600 using a variety of technologies well known in the art. In an exemplary embodiment, a user may enter personal information into the heart rate monitor 600 using the user input device 622, as illustrated in FIG. 6A and 6C. For instance, in calculating target heart rate zones using Karvonen formula, base heart rate, age and gender may be provided to the calculating device. For example, through the user input device 622, the user may enter his age using control 607 and his gender using control 608. The entered data may appear on the display 606 for confirmation. Once the heart rate monitor 600 is worn, the device may automatically calculate resting heart rate using the sensor assembly 603 and display it on the display 606. The display 606 may also display other useful data such as time of day and date.

In another exemplary embodiment, a user may be able to input personal information into and view data generated by the heart rate monitor 600 using external devices. The external device may include, but is not limited to, a personal computer, a cell phone, an ipod®, a Palm® device, or similar electronic devices. Accordingly, the heart rate monitor 600 may be outfitted and its microcontroller 20 may be configured with software to receive data from external devices either through a wire connection or wirelessly.

FIG. 6E, illustrates a side view of the heart rate monitor 600. On the side surface 629 of the heart rate monitor 600 there may be an input/outlet port 617. This input/output port 617 may be configured to allow an external device, such as a personal computer 611, an iPod® 618 or other a personal device, such as a cell phone 612 to connect to the heart rate monitor 600 for uploading or downloading data via a wired interface 610. The input/output port 617, for example, may be a USB, FireWire (IEEE 1394), RS-232, or other standard wired data link. Instead of a wired connection, a wireless connection may be used, such as to connect to a personal computer 611, an ipod® 618 or other a personal device, such as a cell phone 612, via a wireless interface 619 such as an infrared, wi-fi (802.11), Bluetooth or any other well known wireless datalink technology. This capability will allow a user to enter his personal information into a personal computer 611 or ipod® 618 and have the information transmitted to the microcontroller 20 (illustrated in FIG. 1) for processing via a wired connection 610 to the input/output port 617. The information processed by the microcontroller 20 (illustrated in FIG. 1) may then be displayed on the display 606 and/or on the display unit of the personal computer 611 or iPod® 618.

The data input/output port 617 may allow the user to download the measured physiological parameter, such as over the course of a workout, for subsequent processing or study on the external device. In this way, the user may track and record the progress of the workout routines and improvement over the course of a workout regime. With the capability that data can be exchanged between the heart rate monitor 600 and an external device wirelessly, the information recorded on the heart rate monitor 600 during an exercise routine maybe transmitted to and viewed on a cell phone 612. Other wireless hand held devices may also be used. The cell phone 612 maybe also configured to receive and display other information from the heart rate monitor 600. Configuring a cell phone to receive information from the heart rate monitor 600 may be achieved using well known implementation methods, such as using cell phone 612 processor (not shown) readable software instructions stored the memory (not shown) of the cell phone 612.

In another exemplary embodiment, a user's information collected by the heart rate monitor 600 may be sent to an external database 620 using wired or wireless data link connections, for example, for medical purposes. For medical monitoring, a user may wear the heart rate monitor 600 at all times. The data collected by the heart rate monitor 600 maybe periodically sent to an external server 620, such as a hospital medical data server, where it will be permanently recorded and rendered accessible to the user and his physicians.

FIG. 6F illustrates the bottom view of the portable heart rate monitor 600. On its bottom surface 614, the heart rate monitor 600 may include a sensor assembly 603 for monitoring physiological parameters, and a compartment 602 for storing power generating devices 627, such as a battery. FIG. 6G, illustrates a detailed view of an exemplary heart rate monitor 600 embodiment which may include a compartment 602 on its bottom surface 614. This compartment 602 may allow for installing or storing power generating devices 627, such as a battery. This compartment 602 may be covered by a lid 602A to secure the power generating device 627.

FIG. 6H is a three dimensional view of the heart rate monitor 600 embodiment. The casing 621 of the heart rate monitor 600 may be in the shape of a bracelet. This casing 621 may house the display units 601, the user input device 622 (not shown), the sensors assembly 603, the microcontroller 20 (as illustrated in FIG. 1) and the power generating device 627. The casing 621 of the heart rate monitor 600 may have many different designs. It may be designed to be worn on different body parts, such as wrists, fingers, ears, neck, chest or ankles. It may be fashionably designed so that it may match a user's clothing, jewelry or accessories. It may be designed for use by those who are color blind, have weak eyesight or suffer from other disabilities. It may be designed for use by athletes, such as swimmers. For example, the heart rate monitor 600 may be water resistant, water proof or impact resistance.

In an exemplary embodiment, FIG. 61 illustrates a sensor assembly 603 positioned on the bottom surface 614 of the heart rate monitor 600. When the heart rate monitor 600 is placed around a body structure, the sensor assembly 603 which is on the bottom surface 614 of the heart rate monitor 600 will come in contact with that body structure. The sensor assembly 603 detects physiological parameters and sends the collected data to the microcontroller 20 (illustrated in FIG. 1) for processing. The processed data will in turn be displayed by the display units 601.

In one embodiment, the heart rate monitor 600 may use pulse oximetry technology to monitor physiological data such as heart rate and blood oxygen levels. Pulse oximetry has been used for many years as a mechanism to monitor heart rate and oxygen levels in the blood. In pulse oximetry, a light emitter and photo detector are typically placed on either side of a thin structure of the patient's anatomy, usually a fingertip or earlobe, or in the case of a neonate, across a foot, and a light containing both red and infrared wavelengths is passed from one side to the other. Based upon the ratio of changing absorbance of the red and infrared light caused by the difference in color between oxygen-bound (bright red) and oxygen unbound (dark red or blue, in severe cases) blood hemoglobin, a measure of oxygenation (the per cent of hemoglobin molecules bound with oxygen molecules) can be made.

Changing absorbance of each of the two wavelengths may also be measured, allowing determination of the absorbances due to the pulsing arterial blood alone, excluding venous blood, skin, bone, muscle, fat, and (in most cases) fingernail polish. By examining only the varying part of the absorption spectrum (essentially, subtracting minimum absorption from peak absorption), a monitor can ignore other tissues or nail polish and discern only the absorption caused by arterial blood. The monitored signal bounces in time with the heart beat because the arterial blood vessels expand and contract with each heartbeat. By measuring this variation in time, heart rate may also be measured.

The light emitter and photo detector are commercially available components specified for medical purposes. Typically, the light emitters include small light-emitting diodes (LEDs). However, it should be noted that any of a variety of illumination sources operating in the appropriate frequency range will suffice. Such illumination sources may include, for example, LEDs, LCDs, ELDs, etcs. For illustrative purposes disccusion herein will assume a LED emitter source. One LED emits light in the red range, with wavelength of about 660 nm (±15 nm), and the other emits light in the infrared range, about 905, 910, or 940 nm (±15 nm). Absorption at these wavelengths differs significantly between oxyhemoglobin and its deoxygenated form, therefore the oxy/deoxyhemoglobin ratio can be calculated using the ratio of the absorption of the red and infrared lights. The absorbance of oxyhemoglobin and deoxyhemoglobin is the same (“isobestic point”) for the wavelengths of 590 and 805 nm.

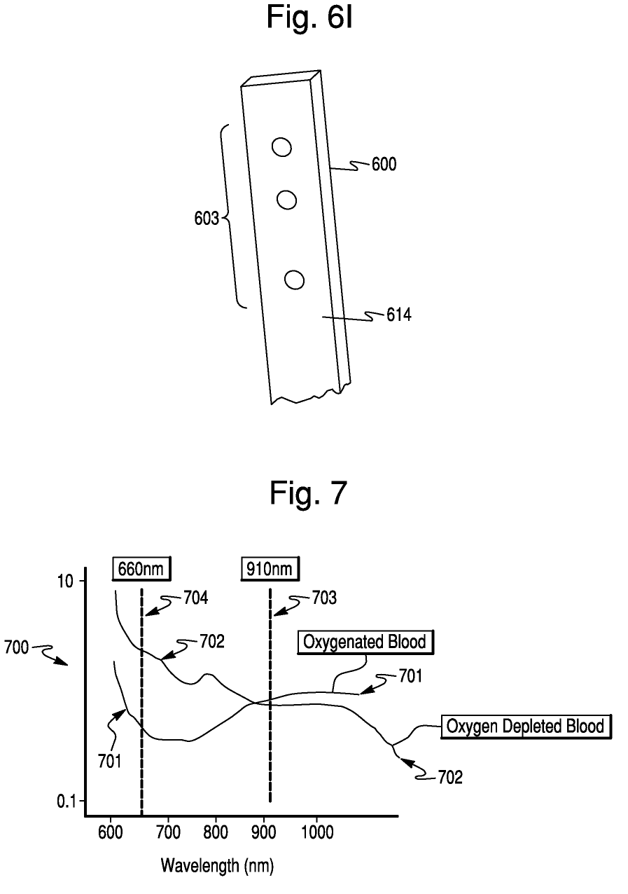

FIG. 7 shows an example of absorption vs. wavelength graph 700. The graph 700 illustrates a large difference in light absorption of oxygneated blood 701 and de-oxygneated blood 702 for light being emitted in the red frequency range of about 660 nm, line 704. In contrast, at the higher infrared frequency of about 910 nm, line 702, there is a small difference between light absorption of oxygenated blood 701 and de-oxygenated blood 702. Therefore, for pulse oximetry calculation purposes, the detected absorption levels of red light, about 660 nm, are used to calculate oxygen levels in the blood. Meanwhile, the detected absorption levels of infrared light, wavelengths of about 910 nm, are used to calculate the pulse or heart beat.

In conventional transmission mode sensor assembly, the light source is positioned opposite of the light detector so that light can travel from an emitter through the tissue and to a photo detector. This only allows the use of such conventional sensors on thin vascular anatomical structures such as the earlobe or fingertip, where light can pass from one side of the anatomical structure to the other, without being blocked by more dense tissue, such as bones and muscles.

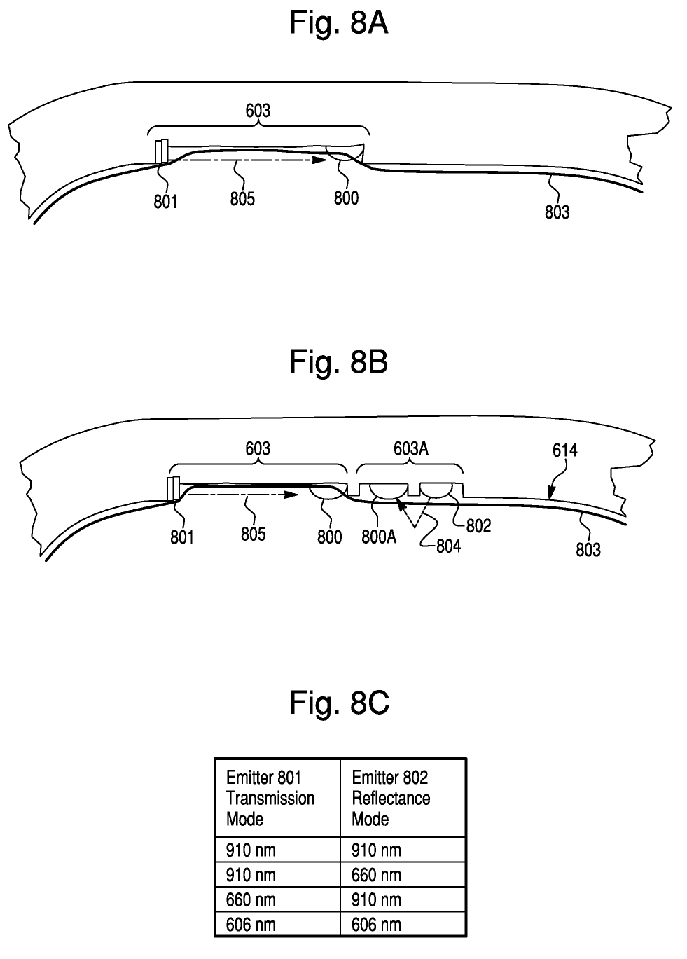

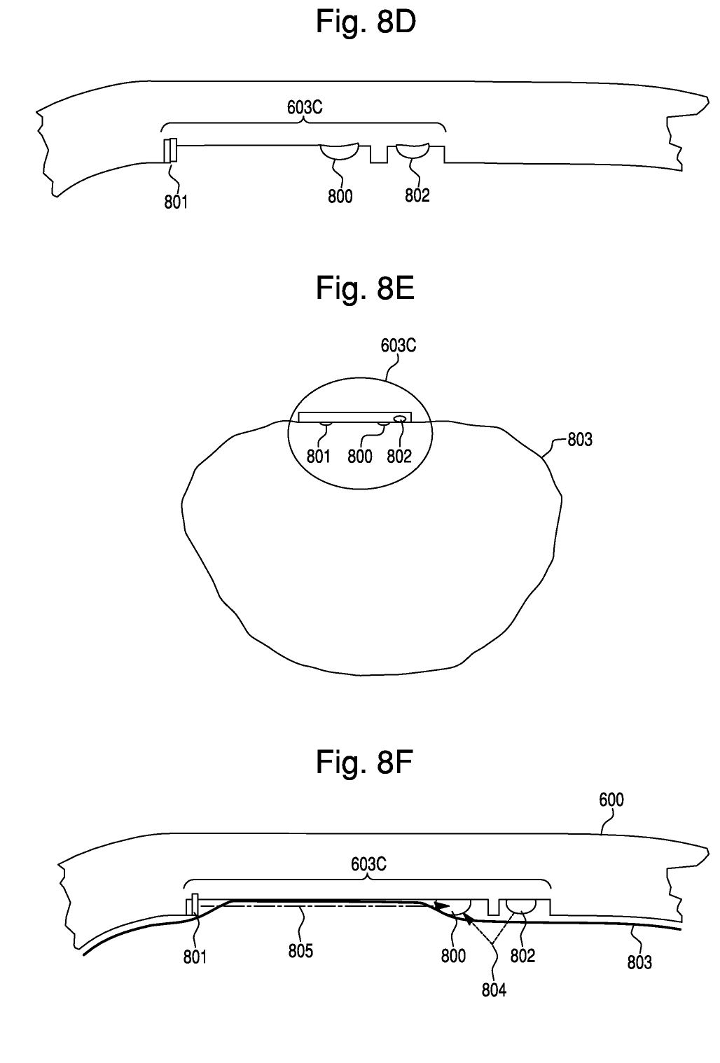

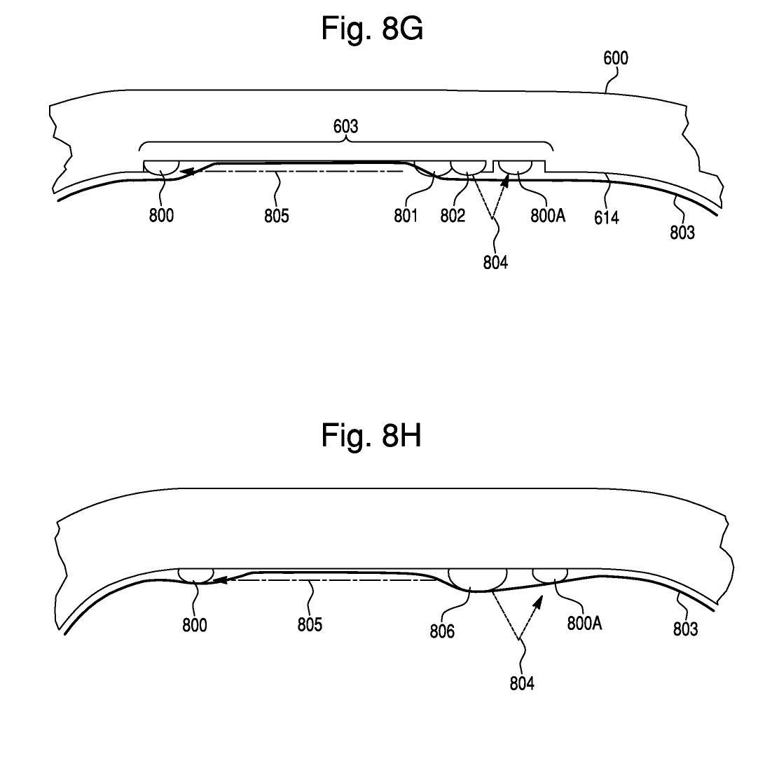

An embodiment overcomes the deficiencies of the conventional transmission mode sensor assemblies by providing the transmission mode sensor assembly in a one-sided arrangement as illustrated in FIG. 8A. FIG. 8A shows a cross-sectional view of the sensor assembly 603 embodiment as illustrated in FIG. 61. In this embodiment, the sensor assembly 603 is positioned on the bottom surface 614 of a heart rate monitor 600 where it can come into contact with a body anatomical structure, such as a wrist 803, when the heart rate monitor 600 is worn. This transmission mode sensor assembly 603 includes an emitter 801, such as an edge emitter, and a photo detector 800. Light 805 from the emitter 801 is transmitted superficially through the tissue of the wrist 803 and received by the photo detector 800. In the one-sided arranged sensors of the embodiment, the emitter 801 and the photo detector 800 are placed side-by-side, creating the one-sided arrangement. This side arrangement will allow the sensors to be useful in monitoring physiological information from all anatomical structures and take many different designs, such as a heart rate monitor 600 in the shape of a bracelet. Data retrieved from the sensor assembly 603, as illustrated in FIG. 8A, is directed to the microcontroller 20 (illustrated in FIG. 1) for processing. The microcontroller 20 (illustrated in FIG. 1) may then convey the processed data to a display unit 601 (illustrated in FIG. 6A).

In an alternative embodiment the transmission sensor assembly 603, embodiment shown in FIG. 8A, is combined with a reflectance sensor assembly 603A. This combination of sensor assemblies 603 and 603A is illustrated in FIG. 8B. Combining the monitoring function of sensor assembly 603 and 603A allows for a more accurate and constant monitoring of the pulse and blood oxygen levels. Accordingly, the heart rate monitor device of the embodiment may utilize transmission mode and/or reflectance mode simultaneously and on dense anatomical structures such as a wrist or neck. By measuring in two different modes from one device, pulse oximetry may be extended to include many more applications and the robustness of existing devices may be improved.

As shown in FIG. 8B, sensor assembly 603 which includes a one-sided transmission sensor assembly and sensor assembly 603A which includes a reflectance sensor assembly may be placed on the bottom surface 614 of the heart rate monitor 600. When the bottom surface 614 of the heart rate monitor 600 comes into contact with an anatomical structure of a user, such as a wrist 803, the sensor assemblies 603 and 603A fall in a position to detect pulse and/or oxygen blood levels. These sensor assemblies 603 and 603A in combination may include two photo detectors 800 and 800A, an emitter 801, such as an edge emitter LED, and an emitter 802, such as a surface emitter LED. The emitter 801 may function in transmission mode and transmit light 805 through an anatomical structure to the photo detector 800. The emitter 802 may function in reflectance mode and transmit light 804 to a photo detector 800A by reflecting the light 804 through the tissue of an anatomical structure, such as a wrist 803. The data received by the photo detectors 800 and 800A is also communicated to the microcontroller 20 (illustrated in FIG. 1) where it is processed and subsequently displayed on the display units 601 (illustrated in FIG. 6A). The emitters 801 and 802 may each emit light in different wavelengths. For example, each emitter may emit light at about 910 nm and/or 660 nm wavelengths.

FIG. 8C shows the possible wavelength combinations for the emitters 801 and 802. As shown in this figure, the first combination of emitters may include both a transmission mode emitter and a reflectance mode emitter emitting infrared light at about 91 0 nm. In this configuration, both sensor assemblies 603 and 603A (illustrated in FIG. 8B) are optimally used to detect heart rate. Alternatively, the transmission mode emitter may be set to emit light in the red range of about 660 nm and the reflectance mode emitter may be set to emit light in the infrared range of about 910 nm or vice versa. In these configurations, the sensor assemblies 603 and 603A (illustrated in FIG. 8B) may be configured to detect both heart rate and oxygen levels as discussed above with respect to FIG. 7. Finally, the first combination of emitters may include both a transmission mode emitter 801 and a reflectance mode emitter 802 emitting infrared light at about 660 nm. In this configuration, both sensor assemblies are optimally used to detect oxygen levels.

In configurations where both the transmission mode emitter 801 and reflectance mode emitter 802 emit light at the same frequency, the configuration allows for a redundancy of detection, thus providing a more robust and accurate reading. In all configurations, light emitted from the emitter 801 and emitter 802 may be detected by the photo detectors 800 and 800A. The collected data is then communicated from the photo detectors 800 and 800A to the microcontroller 20 (illustrated in FIG. 1) for processing and the results are shown to the user through display units 601 (illustrated in FIG. 6A).

In an exemplary embodiment, as illustrated in FIG. 8D, a sensor assembly 603C may function both in one-sided transmission mode and reflectance mode with three sensors. In this embodiment the photo detector 800 functions to receive light from both emitter 801 which functions in transmission mode and emitter 802 which functions in reflectance mode. FIG. 8E illustrates an example of the positioning of a sensor assembly 603 on a wrist 803. For more accurate results, the sensors may be positioned on the vascular part of the wrist 803.

FIG. 8F is a cross-sectional depiction of the sensor assembly 603C of the heart rate monitor 600 as it rests on the wrist 803. In FIG. 8F, the emitter 801 may be placed at a distance from one side of the photo detector 800 to construct a one-sided transmission mode sensor. The light 805 emitted from the emitter 801 passes through the wrist 803, parallel to the surface of the wrist 803, before reaching the photo detector 800. The emitter 802 may be placed at a distance from a second side of the photo detector 800 to construct a reflectance mode sensor. The light 804 moving away from its generating emitter 802, enters the wrist 803 tissue at an angle and is reflected back to the photo detector 800. Data retrieved from the sensor assembly 603C is directed to the microcontroller 20 (illustrated in FIG. 1) for processing. The microcontroller 20 (illustrated in FIG. 1) may then convey the processed data to a display unit 601.

FIG. 8G is an exemplary embodiment illustrating alternative positioning of two sensor assemblies 603, functioning in transmission mode, and 603A functioning in reflectance mode. As shown in FIG. 8G, the emitter 801 and emitter 802 are placed on the bottom surface 614 of the heart rate monitor 600. A photo detector 800 is placed at a position a distance away from the emitter 801 so that the light 805 emitted from emitter 801 passes through the tissue of the wrist 803 and parallel to the surface of the wrist 803, before reaching photo detector 800. The emitter 801 functions in transmission mode. A second photo detector 800A is placed adjacent to emitter 802 so that the light 804 moving away from its generating emitter 802, enters the wrist 803 tissue at an angle and then reflected back to the photo detector 800A. The emitter 802 functions in reflectance mode.

In another exemplary embodiment, as illustrated in FIG. 8H, in instances where the emitter 801 and emitter 802 emit light in the same frequency range, the emitter 801 and emitter 802 may be combined into a single emitter 806. The emitted light from the single emitter 806 is detected in a transmission mode by photo detector 800 and in a reflectance mode by photo detector 800A.

In an exemplary embodiment, the heart rate monitor 600 may employ a variety of currently known technologies, such as embedded radio frequency (RF) receivers or electrocardiography (ECG) sensors. For example, embedded in a chest worn heart rate monitor maybe an RF receiver assembly. The RF receiver assembly can transmit the user's heart rate signal to another device such as a wrist worn heart rate monitor 600 where the results may be displayed. Further, in another exemplary embodiment, the heart rate monitor 600 may include electrodes to detect ECG signals. Heart rate may then be calculated based on the detected ECG signals and the results shown on the display.

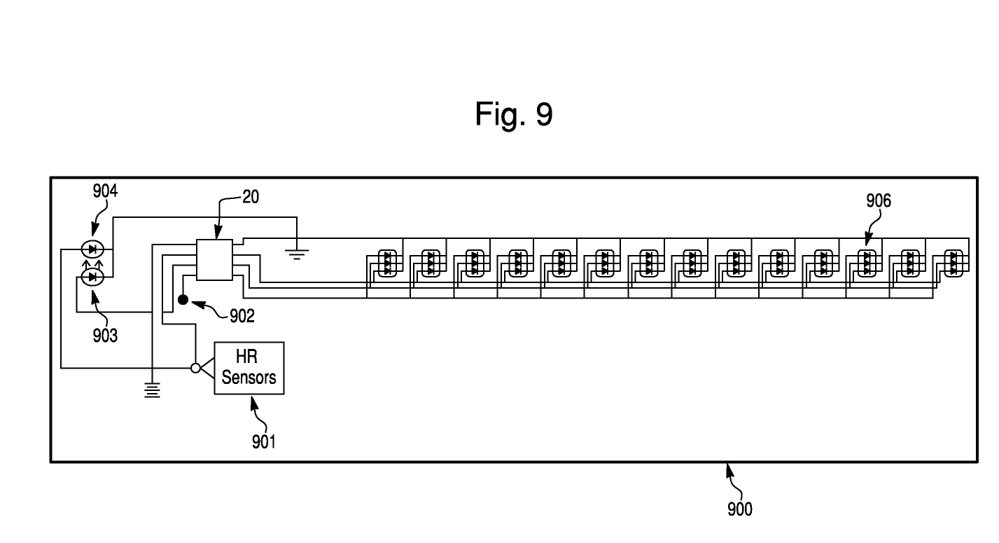

FIG. 9 illustrates an example of a circuit 900. This circuit 900 connects the heart rate sensor assembly 901 to a microcontroller 20. Through this connection, detected data, such as base heart rate, may be communicated from the heart rate sensor assembly 901 to the microcontroller 20 for processing and shown to the user through the illumination color light source display 906. Microcontroller 20 may comprise a application specific integrated circuit (ASIC) or a programmable integrated circuit (PIC) which is specifically designed to control the illumination source display 306. The microcontroller 20 may be programmed to display the spectrum of colors through a blend of three primary color sources as described in more detail below. The circuit 900 may also optionally include illumination color light sources 903 and 904. As shown in FIG. 9, illumination light sources 903 and 904 may be LEDs, but may also be any illumination color light source display 906 operates to change color as the physiological parameter, such as heart rate, changes. For example, as the user's heart rate increases the illumination color light source display 906 may change from blue to yellow to green. Meanwhile, optional illumination color light sources 903 and 904 may operate to blink with the detected pulse rate. Such a detected pulse rate may be detected by heart rate sensor assembly 901 and directly outputted to illumination color light sources 903 and 904. Thus, the user may have an indication of heart rate both by the color shown by illumination color light source display 906 as well as the frequency of the blinking rate of illumination color light sources 903 and 904.

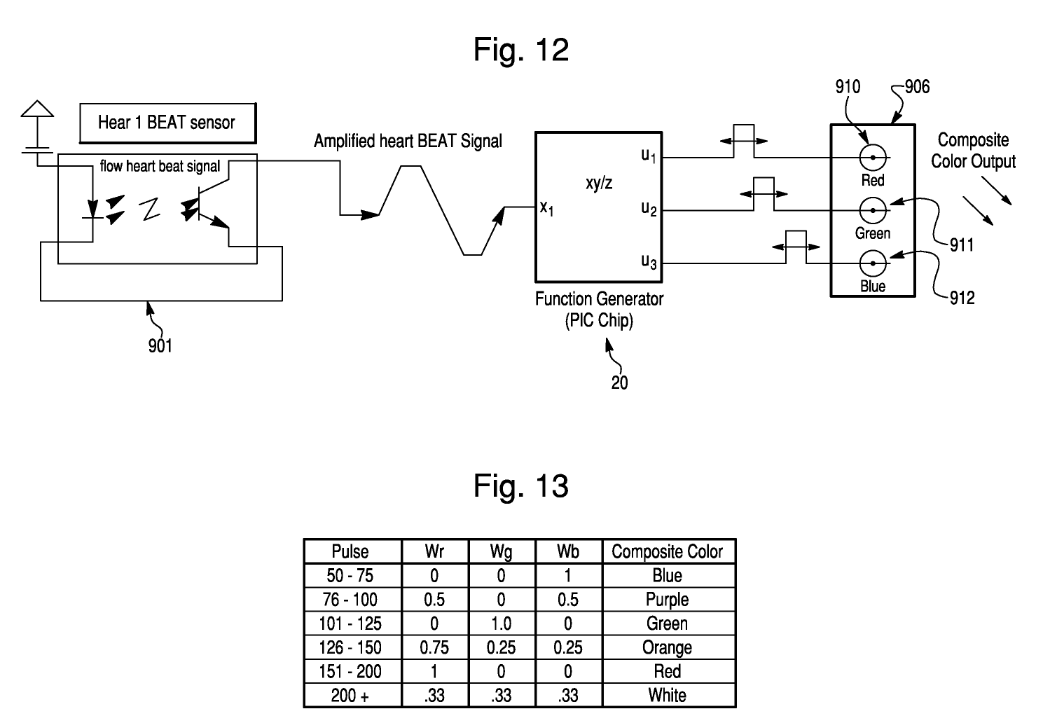

Data gathered from the sensor assembly 901 and input device 904 are processed by the microprocessor 20 using preprogrammed algorithms or formula, such as Karvonen formula. Based on the results of the data processing, the microcontroller 20 selects which color illumination color light source display 906 may be lit to convey that information to the user. FIG. 12 illustrates a detailed view of the sensor assembly 901, microcontroller 20 and illumination color light source display 906. A user's heart rate is detected by the change in light transmission and/or reflectance intensity due to the absorption of the emitted infrared light from emitter, e.g., 801 and 802, when blood is surging through the user's blood vessels near the surface of the user's skin. This detected change in light intensity corresponds to the raw heart beat signal. The raw heart beat signal is outputted from the sensor assembly and amplified by an opto-coupler (not shown) such that an unconditioned modulated analog signal (amplified heart beat signal) is connected to the input X1 of the microcontroller 20. As above, microcontroller 20 may comprise, for example, a ASIC or PIC. The microcontroller 20 receives the low frequency analog signal and multiples it to a usable (varying frequency) digital output signal. The microcontroller 20 may be programmed to varying signals to each output signal which in turn control the three primary (RGB) color light sources 910 (Red), 911 (Green), 912 (Blue) connected to microcontroller 20 and make up the illumination color light source display 906. As is a well known in the area of optics, the three primary colors may be “mixed” to create any color on the spectrum as a “composite” color. Standard program functions of the microcontroller 20 allow the pulse width of each of the three (RGB) outputs to be scaled based on the input frequency. The composite color output of the RGB illumination color light source display 906 can be controlled by varying the ratio of the three individual pulse widths controlling color light sources 910, 911, and 912. Since the frequency of the individual red-green-blue flashes is faster than the human eye can perceive, the eye integrates the pulses into the “composite” color. Such an operation works much the same as mixing pigments of the three primary colors to generate any composite color of the spectrum in paint.

FIG. 13 illustrates an exemplary chart of various pulse widths that may be implemented to achieve a variety of colors outputted by the illumination color light source display 906 which is made up of color light sources 910, 911, and 912. In the first column of the table shown in FIG. 13 indicates a range of pulse rates for a user. For example, in the first row, if the user's pulse rate is between 50 and 75 heart beats per minute, the illumination color light source display 906 may illuminate with a blue color. Thus, as shown in the table of FIG. 13, the microcontroller 20 outputs a signal on the blue output which controls the blue color light source 912 for the entire pulse width duration. Thus, the outputted light signal will be blue. In the second column of the table of FIG. 13, the approximate width of the red output pulse is indicated. The next column indicates the approximate width of the green output pulse. The third column indicates the approximate width of the blue output pulse. The final column indicates the resultant composite color that is effectively generated.

Thus, the pulse rate of the user was detected to be between 76 and 100 beats per minute, the illumination color light source display 906 may illuminate with a purple color. The purple color may be achieved by flashing the red color light source 910 and the blue color light source 912 equally. Thus, as shown in the table of FIG. 13, the microcontroller 20 outputs a signal on the red output for half (0.5) of the pulse width and a signal on the blue output for half (0.5) of a pulse width. The microcontroller 20 does not output any signal on the green output. Since the frequency at which the color light sources 910, 911, and 912 flash is faster than the frequency which can be perceived by the human eye, the perceived color will be purple. As will be easily recognized by one of skill in the art, the actual colors corresponding to various pulse rate ranges, and the limits of those ranges may be modified and customized by the user or programmer of the device.

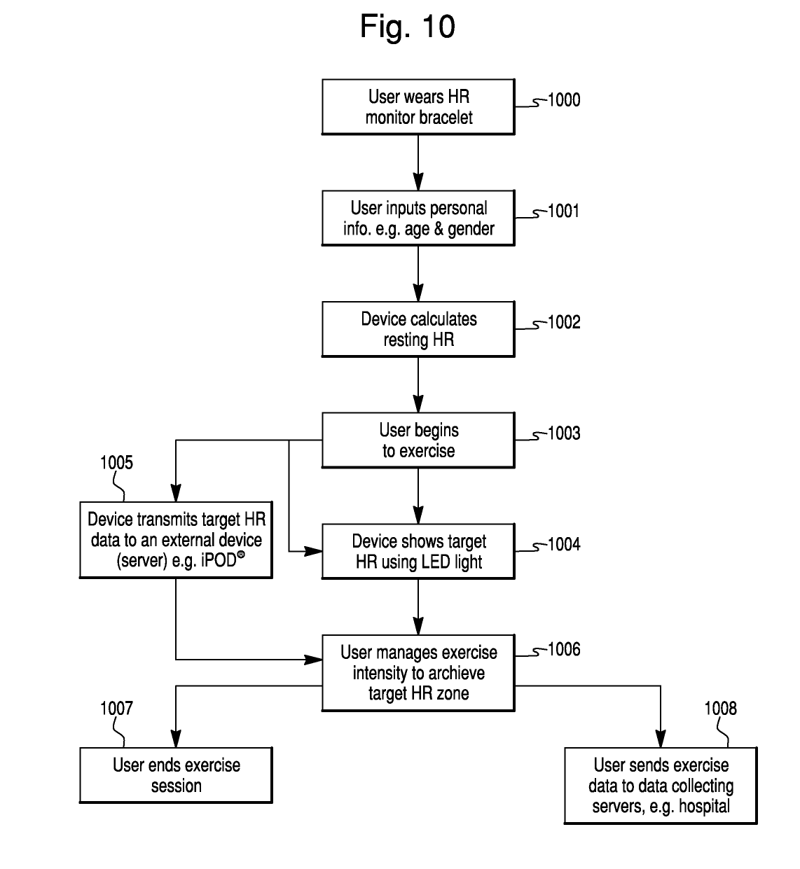

FIG. 10 provides a flow process diagram of the various embodiment methods described above. Referring to FIG. 10, the user attaches the heart rate monitor bracelet to a body part, such as a wrist, ankle, neck, or waist, step 1000. The user inputs his personal information, such as age and gender, into the heart rate monitor, step 1001. This step may be accomplished directly on the device or via a personal computer, ipod®, or some other device connected to the heart monitor bracelet via a wired or wireless connection. In use, the heart rate monitor detects base heart rate of the user, step 1002. Once the user begins exercising, step 1003, the heart rate monitor displays in color the progression of the user's heart rate until it reaches a target heart rate zone, step 1004. For example, yellow may mean that target heart rate has not yet been achieved. Green may mean that target heart rate has been achieved. Red may mean that target heart rate has been passed. The display of color may utilize the LED display 601 which effectively light up the entirety of the heart monitor bracelet for quick and easy reading of heart rate. Alternatively, the data regarding the target heart rate zone may be transmitted to an external device or data collection server, using a wired or wireless connection, step 1005. Based on the information received from the heart rate monitor display lights 601 the user manages his target heart rate by increasing or decreasing the intensity of the exercise, step 1006. Once the user maintains his target heart rate for a predetermined period of time, the user finishes exercising, step 1007. Alternatively, at the end of the exercise session, user can send the generated data to an external storage server using a wired or wireless connections, step 1008.

The device of the various embodiments may be used for purposes other than exercise, such as monitoring of physiological parameters of a patient. This monitoring may occur in the hospital or from a remote location, such as the patient's home. Conventional physiological monitoring devices are cumbersome to wear and hinder the patients' freedom of movement. These devices have many wire connections, must be worn on uncomfortable anatomical structures, such as the finger tip or earlobe and restrict the movement of the patients to a small area which is as long as the connections wires will reach. The physiological monitoring device of the various embodiments may be easy to wear, use and may use wireless connections which do not restrict the patients' movement.

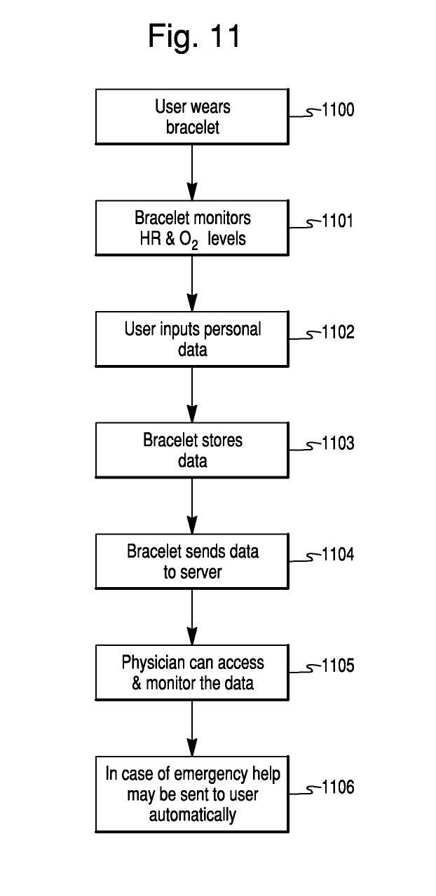

FIG. 11 provides a flow process diagram of the various embodiment methods described above. Referring to FIG. 11, the patient places the physiological monitoring bracelet around a body part, such as a wrist, ankle, neck, or waist, step 1100, and inputs personal data into the bracelet, step 1101. This step may be accomplished directly on the device or via a personal computer, iPod(g), or some other device connected to the heart monitor bracelet via a wired or wireless connection. The bracelet subsequently monitors heart rate and blood oxygen levels, step 1102. The bracelet stores the monitored physiological data to an internal memory unit for temporary storage, step 1103, and/or sends the stored monitored data to an external server either through wired or wireless connections, step 1104. A physician may access and monitor the transmitted data from the server to make an accurate diagnosis and monitoring of a patient's condition, step 1105. In cases where the monitored physiological data indicate imminent danger to the patient, an emergency response team may be automatically alerted and dispatched to the patient's aide, step 1106.

The present heart rate monitor in any of its embodiments enables the user to quickly and easily note the general range of his or her heart rate. The easily viewed color display enables a user or patient to determine the level of their heart rate at a glance. This allows a user who is exercising to determine their heart rate without having to slow or stop the exercise activity to read and interpret a relatively small digital display, as is conventionally found in other heart rate indicating devices. The present heart rate monitor will also be beneficial to those persons who require corrective lenses, but who do not wear them during exercise. The easily viewed color display of the present heart rate monitor enables those persons with less than perfect eyesight, to note their general heart rate without need for any supplemental vision correction while exercising. The ease of comprehension of the present heart rate monitor will enable users to make better progress toward achieving their goals of better fitness and weight loss. As the colors provided by the display of the present heart rate monitor relate directly to established nomenclature and exertion levels, increased motivation and feedback is provided for users to enable them to improve their performance and achieve their goals. As the primary information required of most persons while exercising is their general heart rate range, and the knowledge that their heart rate (and thus their level of exertion) is appropriate for their condition, the present heart rate monitor in any of its embodiments will prove to be most beneficial to the average person who wishes to maintain their health.

It is to be understood that the present invention is not limited to the embodiments described above, but encompasses any and all embodiments within the scope of the following claims.

We claim:

1. A device for monitoring body physiology parameters, comprising:
a casing;
a sensor assembly positioned within the casing, the sensor assembly configured to monitor body physiological parameters and comprising
an emitter emitting light in transmission mode, and
a photo detector receiving light from the emitter and positioned to the side of the emitter for a one-sided light transmission;
a microcontroller positioned within the casing and configured to transmit data to and communicate with the sensor assembly; and
a display positioned within the casing and configured to display information and communicate with the microcontroller.

2. The device for monitoring body physiology parameters as claimed in claim 1, wherein the display comprises a series of multi-color illumination sources covering an entirety of the device.

3. The device for monitoring body physiology parameters as claimed in claim 1, wherein the sensor assembly includes an ECG electrode.

4. The device for monitoring body physiology parameters as claimed in claim 1, wherein the sensor assembly includes a Radio Frequency receiver.

5. The device for monitoring body physiology parameters as claimed in claim 2, wherein the emitter is an edge emitter illumination source.

6. The device for monitoring body physiology parameters as claimed in claim 2, wherein the body physiology parameter is heart rate and the emitter emits light with wavelength of about 910 nm.

7. The device for monitoring body physiology parameters as claimed in claim 2, wherein the body physiology parameter is blood oxygen levels and the emitter emits light with wavelength of about 660 nm.

8. The device for monitoring body physiology parameters as claimed in claim 2, wherein the microcontroller is configured to calculate target heart zone using formulas which calculate physiological parameters and convey that information to the display.

9. The device for monitoring body physiology parameters as claimed in claim 7, wherein the microcontroller is configured to receive blood oxygen measurements and convey that information to the display.

10. The device for monitoring body physiology parameters as claimed in claim 2, wherein the microcontroller is configured to store monitored physiological information.

11. The device for monitoring body physiology parameters as claimed in claim 10, wherein the microcontroller is configured to send monitored information to an external device connected to the device for monitoring body physiology by wire.

12. The device for monitoring body physiology parameters as claimed in claim 10, wherein the microcontroller is configured to send monitored information to an external device wirelessly.

13. The device for monitoring body physiology parameters as claimed in claim 11, wherein the external device is a computer server.

14. The device for monitoring body physiology parameters as claimed in claim 2, wherein the microcontroller is configured to determine which colors are displayed in accordance with formulas which calculate physiological parameters.

15. The device for monitoring body physiology parameters as claimed in claim 15, wherein the microcontroller is configured to determine which colors are displayed in accordance with the information received from the photo detector.

16. A device for monitoring body physiology parameters as claimed in claim 1, wherein the casing is selected from a group consisting of a bracelet, finger ring, necklace, glasses, chest strap and anklet.

17. A device for monitoring body physiology parameters, comprising:
a casing;
a sensor assembly, placed within the casing, configured to monitor body physiological parameters and comprising,
a first emitter emitting light in transmission mode,
a second emitter emitting light in reflectance mode, and
a photo detector positioned to the side of and configured to receive light from the first and second emitters;
a microcontroller, positioned within the casing, configured to process and transmit data and communicate with the sensor assembly;
a display, positioned within the casing, configured to display information and communicate with the microcontroller; and

18. The device for monitoring body physiology parameters as claimed in claim 17, wherein the display comprises a series of multi-color illumination sources covering an entirety of the device.

19. The device for monitoring body physiology parameters as claimed in claim 18, wherein the sensor assembly includes an ECG sensor.

20. The device for monitoring body physiology parameters as claimed in claim 18, wherein the sensor assembly includes a Radio Frequency sensor.

21. A device for monitoring body physiology parameters as claimed in claim 18, wherein the emitter is an edge emitter illumination source.

22. A device for monitoring body physiology parameters as claimed in claim 18, wherein the body physiology parameter is heart rate and the first emitter and the second emitter emit light at a wavelength of about 910 nm.

23. A device for monitoring body physiology parameters as claimed in claim 18, wherein the body physiology parameter is blood oxygen levels and the first and the second emitters emit light with wavelength of about 660 nm.

24. A device for monitoring body physiology parameters as claimed in claim 18, wherein the body physiology parameters are heart rates and blood oxygen levels and the first emitter and the second emitter emit light with a wavelength selected from a group of about 660 nm and 910 nm.

25. A device for monitoring body physiology parameters as claimed in claim 18, wherein the microcontroller is configured to receive heart rate measurements and convey that information to the display.

26. A device for monitoring body physiology parameters as claimed in claim 22, wherein the microcontroller is configured to calculate target heart zone using formulas which calculate physiological parameters and convey that information to the display.

27. A device for monitoring body physiology parameters as claimed in claim 23, wherein the microcontroller is configured to receive blood oxygen measurements and convey that information to the display.

28. A device for monitoring body physiology parameters as claimed in claim 24, wherein the microcontroller is configured to receive heart rate and blood oxygen level measurements and convey that information to the display.

29. A device for monitoring body physiology parameters as claimed in claim 18, wherein the microcontroller is configured to store monitored physiological information.

30. A device for monitoring body physiology parameters as claimed in claim 27, wherein the microcontroller is configured to send monitored information to an external device connected to the device for monitoring body physiology by wire.

31. A device for monitoring body physiology parameters as claimed in claim 27, wherein the microcontroller is configured to send monitored information to an external device wirelessly.

32. A device for monitoring body physiology parameters as claimed in claim 29, wherein the external device is a computer server.

33. A device for monitoring body physiology parameters as claimed in claim 30, wherein the external device is selected from a group consisting of personal computers, mobile phones and hand held devices.

34. A device for monitoring body physiology parameters as claimed in claim 30, wherein the external device is an external computer server.

35. A device for monitoring body physiology parameters as claimed in claim 18, wherein the display includes an illumination source for displaying information in a multitude of colors.

36. A device for monitoring body physiology parameters as claimed in claim 34, wherein the microcontroller is configured to determine which colors are displayed in accordance with formulas which calculate physiological parameters.

37. A device for monitoring body physiology parameters as claimed in claim 34, wherein the microcontroller is configured to determine which colors are displayed in accordance with the information received from the photo detector.

38. A device for monitoring body physiology parameters as claimed in claim 18, wherein the casing is selected from a group consisting of a bracelet, finger ring, necklace, glasses, chest strap and anklet.

39. A method for using a device for monitoring physiological parameters, comprising:
securing the device on an anatomical structure to bring a sensor assembly adjacent to the skin of a user;
inputting personal information into a user input device, wherein the user input device is in communication with a microcontroller;
determining resting heart rate of the user by using the sensor assembly in communication with the microcontroller;
monitoring physiological parameters during exercise as they are sensed by the sensor assembly and the results are communicated to a microcontroller;
storing the monitored variable physiological parameters during exercise using the microcontroller;
calculating a result using the recorded variable physiological parameters using the microcontroller configured to use an algorithm; and
displaying the result using colored lights, wherein the display receives data for displaying from the microcontroller.
